SEQUENCE LISTING (10 sequences)

SEQ ID NO: 1 - skipped
  RRSFL

SEQ ID NO: 2 - protein, 5 aa, artificial sequence
  synthetic peptide
RRSFV

SEQ ID NO: 3 - protein, 5 aa, artificial sequence
  sythetic peptide
RRASL

SEQ ID NO: 4 - protein, 5 aa, artificial sequence
  synthetic peptide
RRFSV

SEQ ID NO: 5 - protein, 5 aa, artificial sequence
  synthetic peptide
RRMSV

SEQ ID NO: 6 - protein, 5 aa, artificial sequence
  synthetic peptide
RRMTV

SEQ ID NO: 7 - protein, 4 aa, artificial sequence
  synthetic peptide
RMSF

SEQ ID NO: 8 - protein, 4 aa, artificial sequence
  synthetic oligonucleotide
RRSF

SEQ ID NO: 9 - protein, 4 aa, artificial sequence
  synthetic peptide
RRMS

SEQ ID NO: 10 - protein, 7 aa, artificial sequence
  synthetic peptide
LRRASLG

(19) United States
(12) Patent Application Publication
(10) Pub. No.: US 2015/0315569 A1
(43) Pub. Date: Nov. 5, 2015

(54) METHOD FOR SCREENING A NUCLEIC ACID-PROGRAMMED SMALL MOLECULE LIBRARY

(71) Applicant: The Board of Trustees of the Leland Stanford Junior University, Stanford, CA (US)

(72) Inventors: Rebecca Maile Weisinger, Belmont, CA (US); Pehr A.B. Harbury, San Francisco, CA (US); Patrick O. Brown, Stanford, CA (US)

(21) Appl. No.: 14/696,290

(22) Filed: Apr. 24, 2015

Related U.S. Application Data
(60) Provisional application No. 61/987,675, filed on May 2, 2014.

(51) Int. Cl. C12N 15/10; C07K 7/06; C07K 5/11

(52) U.S. Cl. CPC C12N 15/1065; C12N 15/1058; C07K 5/1019; C07K 7/06; A61K 38/00

(57) ABSTRACT

Provided herein is method of screening, comprising: a) combining a nucleic acid-programmed small molecule library with: an enzyme, and a substrate for the enzyme, wherein each of the members of the library comprises a test agent that is linked to an nucleic acid tag that encodes the test agent and the combining results in transferring a chemoselective functional group from the substrate onto at least some of the members of the library; b) isolating the library members onto which the chemoselective functional group has been covalently transferred; and c) amplifying the nucleic acid tags of the library members isolated in step b) to produce an amplification product. Libraries and kits for performing the method are also provided as are compounds and pharmaceutical compositions thereof.

### CROSS-REFERENCING

This application claims the benefit of U.S. Provisional Application Ser. No. 61/987,675, filed May 2, 2014, which application is incorporated herein in its entirety.

### GOVERNMENT RIGHTS

This invention was made with Government support under contract OD000429 awarded by the National Institutes of Health. The Government has certain rights in the invention.

### BACKGROUND

Knowledge of the complete molecular inventory of organisms, and the ability to comprehensively measure and manipulate their gene expression, has been a key driver of biological discovery over the last fifteen years. It remains difficult, however, to control post-translational events. A fundamental current challenge is to identify proteome-wide sets of small molecules that can modulate function, or simply act as affinity reagents. Compounds with the ability to influence protein-protein interactions (for example, cyclosporine and rapamycin), or to recognize compact molecular features (for example, vancomycin), are especially attractive. A reliable way to engineer small molecules with these capabilities would address an existing experimental bottleneck, and open new biological frontiers for exploration.

Directed in vitro chemical evolution is an emerging small-molecule discovery technology that, in principle, can be used to prepare complex libraries of organic molecules of low molecular weight. Such in vitro molecular libraries are generally assembled from a large synthon alphabet by combinatorial chemistry.

### SUMMARY

Provided herein is a method of screening, comprising: a) combining a nucleic acid-programmed library with: (i) an enzyme, and (ii) a substrate for the enzyme; wherein each library member comprises a test agent that is linked to an nucleic acid tag that encodes the test agent and where the enzyme covalently transfers a chemoselective functional group from the substrate to one or more library members; b) isolating the library members onto which the chemoselective functional group has been covalently transferred; and c) amplifying the nucleic acid tags of the library members isolated in step b) to produce an amplification product. In some embodiments, tags of the library members isolated in step b) may be subjected to molecular evolution. In these embodiments, the amplifying step may comprise mutating and/or recombining the nucleic acid tags of the library members isolated in step b) with one another to produce new nucleic acid tags.

In some embodiments, the nucleic acid tags of the library members isolated in step b) are optionally recombined with one another to produce new nucleic acid tags.

In some embodiments, the method may further comprise: d) making a second nucleic acid-programmed small molecule library using the amplification product of c) or diversified progeny of product c) generated by mutation of members of c) and/or by recombination between members of c); e) combining the second nucleic acid-programmed small molecule library with: (i) the enzyme, and (ii) a substrate for the enzyme; wherein where the enzyme covalently transfers a chemoselective functional group from the substrate to one or more library members; f) isolating the library members onto which the chemoselective functional group has been covalently transferred in step e); and g) amplifying the nucleic acid tags of the library members isolated in step f).

In certain embodiments, the method may comprise subjecting the library to consecutive rounds of capture and resynthesis (i.e., by successively repeating steps d) to g) one or more times) to produce a final amplification product. In some embodiments, the method may comprise sequencing the final amplification product, thereby identifying a test agent onto which the chemoselective functional group has been attached.

In some embodiments, the enzyme may transfer a chemoselective functional group from the substrate onto at least some of the members of the library. In these embodiments, the transferred chemoselective functional group may be a thiol group; however a variety of different chemistries can be used. In some embodiments, the transferred chemoselective functional group can be a dipolarophile or a dipolar, such as an azide or alkyne group, which can participate in click reactions. In some embodiments, the substrate is gamma-thio-ATP, although a variety of different substrates can be used.

In these embodiments, the method may comprise reacting the chemoselective functional group with a capture molecule, and then isolating the library members onto which the chemoselective functional group has been covalently transferred. In some embodiments, the capture molecule may be bound to a substrate such as a bead or the like. In one case, the capture molecule may comprise a biotin moiety and a site that is reactive with the chemoselective functional group.

In some embodiments, the enzyme may be a kinase, although the method may be done using a variety of different enzymes.

The nucleic acid-programmed small molecule library may have a complexity of at least 10³, e.g., at least 10⁴, at least 10⁵, at least 10⁶, at least 10⁷, at least 10⁸, at least 10⁹ or at least 10¹⁰, etc. The test agents in the library may be of at least 4 residues (e.g., at least 5 residues at least 6 residues or at least 7 or more, etc.) residues in length.

Also provided herein is a method for splitting a nucleic acid-programmed small molecule library into two or more parts such that all the parts of the library contain essentially the same test agents. In some embodiments, this method may involve: a) making a nucleic acid-programmed small molecule library that comprises at least a first set of members and a second set of members, wherein the first set of members and the second set of members are essentially identical except for a tag that allows the first and second sets of members to be separated from one another by hybridization; and b) separating the first and second sets from one another by hybridization. One of the parts of the library may be used as a control for the other. In some embodiments, the method may further comprise: c) screening the first set of library members under a first set of conditions to obtain first results; d) screening the second set of library members under a second set of conditions to obtain second results; and e) comparing the results obtained from steps c) and d). In some embodiments, the initial library composition may comprise: a) a first set of members of a nucleic acid-programmed small molecule library; and b) a second set of members of a nucleic acid-programmed small molecule library, wherein the first and second sets of members of the library are essentially identical except for a tag that allows the first and second sets of members to be separated from one another by hybridization.

In some embodiments, a method of measuring gene enrichment is provided. The method comprises: a) making a nucleic acid-programmed small molecule library that comprises at least a first set of members and a second set of members, wherein the first set of members and the second set of members are essentially identical except for a sequence tag that allows the first and second sets of members to be separated from one another by hybridization; b) screening the first set of library members under a first set of conditions to obtain first results (e.g., using the method described above to obtain a first set of library members onto which a chemoselective functional group has been transferred by an enzyme) and not screening the second set of library member; (c) separating the first and second sets from one another by hybridization to the sequence tag; and (d) calculating the ratio of a gene's fractional abundance in the first set of library members relative to its fractional abundance in the second population.

Also provided are a variety of kits. In some embodiments, a kit may comprise a) a nucleic acid-programmed small molecule library, wherein each of the members of the library comprises a test agent that is linked to an nucleic acid tag that encodes the test agent; b) an enzyme; and c) a substrate for the enzyme, wherein the enzyme covalently transfers a chemoselective functional group from the substrate onto at least some of the members of the library. The kit may further comprise a capture molecule that reacts with the chemoselective functional group. In some embodiments, the capture molecule may contain a biotin moiety.

In an enzymatic labeling step, a chemoselective functional group may be enzymatically transferred from a substrate to a test agent of the library. Any convenient enzyme may be utilized in the subject methods. Enzymes of interest include, a kinase, a phosphatase, a hydrolase, a glycosidase, a lipase, a fatty acid ligase (e.g., lipoic acid ligase) an esterase, a protease, a ubiquitin tagging enzyme, or any convenient enzyme that finds use in a post-translational modification (e.g., methylation, phosphorylation, ubiquitinylation, N-methylation, O-glycosylation, N-glycosylation, etc).

In some embodiments, the enzyme is a kinase capable of transferring a phosphate group to a substrate to a compound. In certain embodiments, the kinase is used in conjunction with a modified substrate, e.g., thiophosphate-modified substrate, such that a functional group is transferred from the substrate to the compound. The compound may then be chemically labeled via the introduced functional group (e.g., a thiophosphate group or a phosphonate group).

In some embodiments, the enzyme is an enzyme that is capable of acylating a compound with an acyl substrate. Any convenient acyltransferase enzymes may be utilized. Acyltransferase enzymes of interest include, but are not limited to, histone acetyltransferases (HAT), lipases, fatty acid lipoic acid lipase, and the like. The acyltransferase may transfer a chemoselective functional group or a reporter tag from a modified substrate to the compound of interest.

The substrate for the enzyme may contain or may be modified to contain a chemoselective functional group and the enzyme catalyzes the transfer of the chemoselective functional group onto the test agent. Substrates of interest include, but are not limited to, a nucleotide phosphate (e.g., ATP, ADP or an AMP), a sugar, a fatty acid, a peptide. In some embodiments, the substrate includes a thiophosphate. In certain embodiments, the substrate is a thiolated ATP. In some embodiments, the substrate is azido-modified. In certain embodiments, the substrate is an azido-modified ATP, such as 2-azido-ATP or 8-azido-ATP. In certain embodiments, the substrate is an azido-modified fatty acid (e.g., 10-azidododecanoic acid, a substrate for the enzyme lipoic acid ligase) or an azido-modified acetyl substrate, or an azido-modified acyl group. In some embodiments, the substrate is an azido-containing sugar. In certain embodiments, the substrate is modified with an alkynyl group.

Also provided are compounds selected from the group consisting of: RRSFL (SEQ ID NO:1), RRSFV (SEQ ID NO:2), RRASL (SEQ ID NO:3), RRFSV (SEQ ID NO:4), RRMSV (SEQ ID NO:5), RRMTV (SEQ ID NO:6), RMSF (SEQ ID NO:7), RRSF (SEQ ID NO:8) and RRMS (SEQ ID NO:9). The compounds may be combined with a pharmaceutically acceptable excipient to provide a pharmaceutical composition.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates a member of a nucleic acid-programmed small molecule library.

FIG. 2 schematically illustrates a tag of a member of a nucleic acid-programmed small molecule library.

FIG. 3 illustrates a library structure. Genes that program small-molecule synthesis comprise four chemistry-coding regions (VA-VD, rainbow bars) with 384 distinct DNA codon sequence variants possible at each region (totaling 1536 codons). The codons direct addition of seventeen different Fmoc-protected amino acids. An arginine dimer is included as an 18th amino acid in the fourth and final synthetic step. An extra bar code (VE, black/white bar) specifies whether the gene product will be subject to a PKA substrate selection or to a mock selection. The encoded peptide is coupled to the gene through a 5′ polyethylene glycol linker.

FIG. 4 illustrates chemical translation. The DNA genes are split into 384 sub-pools by hybridization of the codons in one coding region to a spatially arrayed set of complementary oligonucleotides. The DNA genes are then transferred in a one-to-one fashion to a 384-well filter plate loaded with DEAE-Sepharose, which acts as a solid support during chemical coupling steps. One of seventeen different Fmoc-protected amino acids (dependent on the sub-pool position within the 384-well plate) is coupled to the growing peptide chain linked toDNA. After the chemical step, the genes are pooled, and the process is repeated until all of the coding regions have been chemically translated.

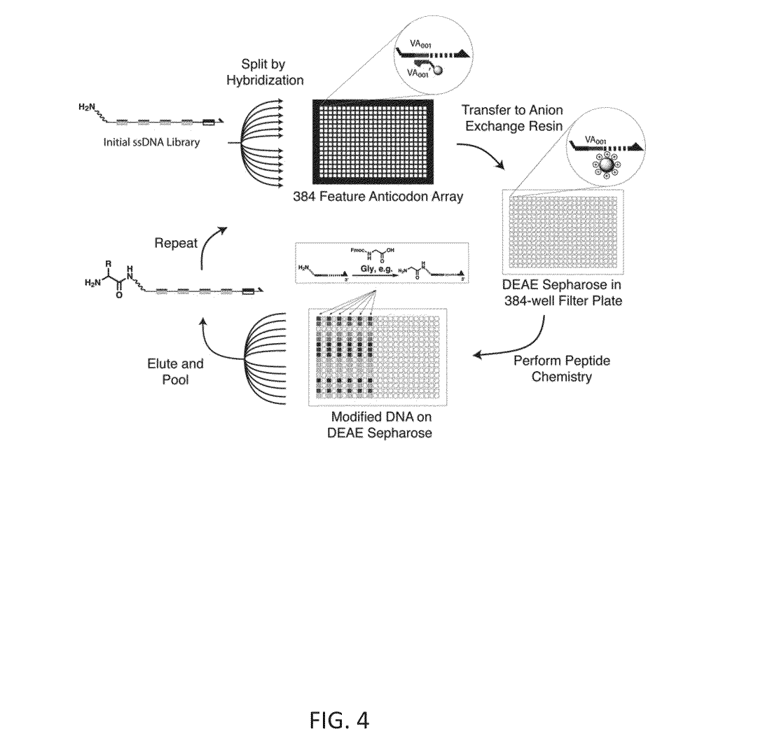

FIG. 5 illustrates directed evolution of kinase substrates. An initial population of DNA genes are chemically translated into peptide-DNA conjugates, and then treated with protein kinase A. Phosphorylated molecules are isolated. The associated genes are amplified by the polymerase chain reaction, and used to program synthesis of the next library generation. After multiple rounds of substrate maturation, the gene population is sequenced. Individual peptides encoded by enriched genes are synthesized without the DNA tag and tested for their ability to function as kinase substrates.

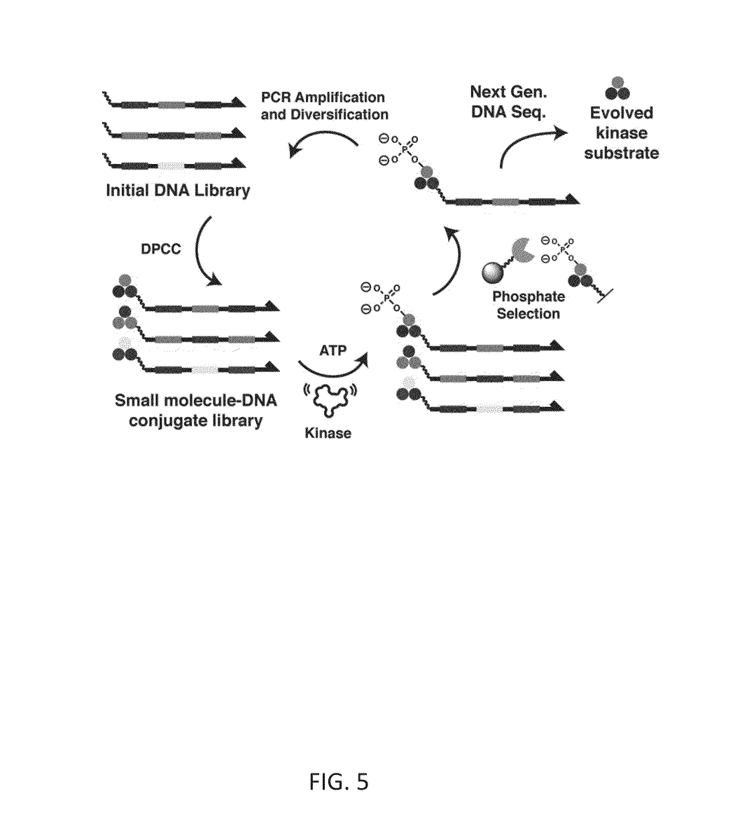

FIG. 6 illustrates phosphate-specific pull-down. The peptide-DNA library was first incubated with protein kinase A and ATP-γ-S. The crude reaction was then treated with the alkylation reagent biotin iodoacetamide, so that thiophosphorylated peptides would become covalently linked to a biotin moiety. Biotinylated molecules were then affinity purified on paramagnetic streptavidin beads.

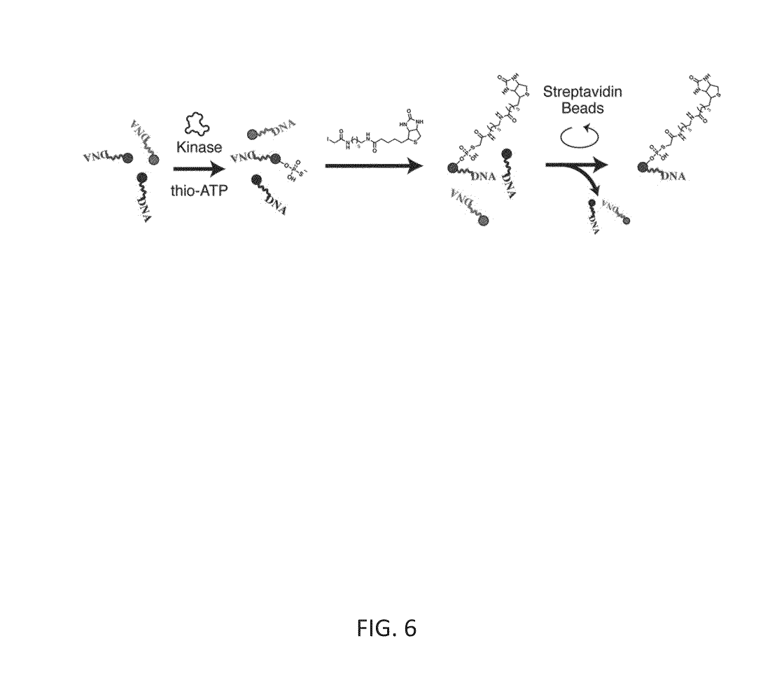

FIG. 7 illustrates population dynamics and genetic noise. A. Selective sweep of the chemical population by PKA substrates. A histogram of the fold-enrichment ratios for the top 1000 genes in generations 2-4 is shown. Genes encoding the most fit peptide are shown in magenta, genes encoding peptides with one of the two consensus motifs are shown in cyan, and genes without a consensus motif are shown in black. B. Suppressing genetic noise. A histogram of the fold-enrichment ratios for 830 genes encoding the most fit peptide is shown (red, 13.9-fold differences cover ±2σ). Narrower distributions indicate a smaller influence of the DNA gene on calculated enrichment.

Two strategies were explored for reducing the influence of gene sequence on the apparent fitness of the gene product. The first strategy was to correct the enrichment ratio of each gene for the systematic drift in underlying codon abundance. This was achieved by calculating enrichment ratios relative to the codon abundance in the mock selection (green, 7-fold differences cover ±2σ), rather than relative to the codon abundance in the initial DNA population (red). The second strategy was to assign multiple codons to each chemical synthon, in order to average out the effects of codon sequence. The distribution of enrichment ratios narrowed when two codons were assigned to each chemical synthon (yellow, 8-fold differences cover ±2σ) relative to the distribution with only one codon assigned to each synthon (red). Applying both strategies gave the narrowest distribution (blue, 4.7-fold differences cover ±2σ), and the smallest influence of the DNA gene on calculated enrichment. C. Specificity and sensitivity of hit detection. Receiver-operating characteristic (ROC) curves show where the genes coding for the best peptide hits (top 60 part per billion in fitness) were located within the list of the top 1 part per million of genes ranked by enrichment ratio. The X-axis shows the fraction of genes on the ranked list that have been tested, and the Y-axis shows the fraction of peptide hits discovered, as one moves successively down the ranked list.

If gene rank correlated perfectly with peptide fitness, all of the genes coding for the best peptide hits would have been at the top of the list. In this ideal case, the curve would go straight up the Y-axis and then cut right on the X-axis at the top of the plot. The yellow and blue curves correspond to two codons per amino acid, and the red and green curves correspond to one codon per amino acid (the same color scheme as in panel B). Enrichments were either corrected for the systematic drift in underlying codon abundance (diamonds), or left uncorrected (circles). A four-read cutoff was applied to genes/gene sets in order to improve the signal-to-noise ratio in the enrichment rankings D. Incremental convergence. ROC curves, as in C, show the position of hits within the ranked gene list over multiple generations.

### DEFINITIONS

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with general dictionaries of many of the terms used in this disclosure. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

The term “combinatorial library” is defined herein to mean a library of molecules containing a large number, typically between 10³ and 10¹⁹ or more different compounds typically characterized by different sequences of subunits, or a combination of different side chains functional groups and linkages.

The terms “base-specific duplex formation” or “specific hybridization” refer to temperature, ionic strength and/or solvent conditions effective to produce sequence-specific pairing between a single-stranded oligonucleotide and its complementary-sequence nucleic acid strand, for a given length oligonucleotide. Such conditions are preferably stringent enough to prevent or largely prevent hybridization of two nearly-complementary strands that have one or more internal base mismatches. Preferably the region of identity between two sequences forming a base-specific duplex is greater than about 5 bp, more preferably the region of identity is greater than 10 bp.

The term “different-sequence small-molecule compounds” refers to small organic molecules, typically, but not necessarily, having a common parent structure, such as a ring structure, and a plurality of different R group substituents or ring-structure modifications, each of which takes a variety of forms, e.g., different R groups. Such compounds are usually non-oligomeric (that is, do not consist of sequences of repeating similar subunits) and may be similar in terms of basic structure and functional groups, but vary in such aspects as chain length, ring size or number, or patterns of substitution.

The term “chemical reaction site” as used herein refers to a chemical component of a nucleic acid tag capable of forming a variety of chemical bonds including, but not limited to; amide, ester, urea, urethane, carbon-carbonyl bonds, carbon-nitrogen bonds, carbon-carbon single bonds, olefin bonds, thioether bonds, and disulfide bonds.

The terms “nucleic acid tag”, “nucleic acid support”, “synthesis-directing nucleic acid tags”, and “DNA-tag” as used herein mean the nucleic acid sequences which each comprise at least (i) a different first hybridization sequence, (ii) a different second hybridization sequence, and (iii) a chemical reaction site. The “hybridization sequences” refer to oligonucleotides comprising between about 3 and up to 50, and typically from about 5 to about 30 nucleic acid subunits. Such “nucleic acid tags” are capable of directing the synthesis of the combinatorial library of the present invention based on the catenated hybridization sequences.

The terms “oligonucleotides” or “oligos” as used herein refer to nucleic acid oligomers containing between about 3 and up to about 200, e.g., from about 5 to about 100 nucleotide subunits. In the context of oligos (e.g., hybridization sequence) which direct the synthesis of the library compounds of the present invention, the oligos may include or be composed of naturally-occurring nucleotide residues, nucleotide analog residues, or other subunits capable of forming sequence-specific base pairing, when assembled in a linear polymer, with the proviso that the polymer is capable of providing a suitable substrate for strand-directed polymerization in the presence of a polymerase and one or more nucleotide triphosphates, e.g., conventional deoxyribonucleotides. A “known-sequence oligo” is an oligo whose nucleic acid sequence is known.

The terms “capture nucleic acid”, “capture oligonucleotide”, “and immobilized capture nucleic acid” as used herein refer to a nucleic acid sequence that is complementary to one of the different hybridization sequences (e.g., a₁, b₁ c₁, etc.) of the nucleic acid tags and therefore allows for sequence-specific splitting of a population of nucleic acid tagged molecules into a plurality of sub-populations of distinct nucleic acid tagged molecules.

The term “non-specific binding” as used herein with respect to a “non-specific filter” refer to binding of nucleic acid that does not depend on the nucleic acid sequence applied to the filter. Exemplary materials for non-specific binding include an ion-exchange medium, which is effective to non-specifically capture nucleic acid tagged molecules at one ionic strength, and release the nucleic acid tagged molecules, following molecule reaction, at a higher ionic strength.

The terms “nucleic acid tag-directed synthesis” or “tag-directed synthesis” or “chemical translation” refer to synthesis of a plurality of compounds based on the catenated hybridization sequences of the nucleic acid tags according to the methods of the present invention.

The terms “tagged compounds”, “DNA-tagged compound”, or “nucleic acid-tagged compound” and grammatical equivalents thereof are used to refer to compounds containing (a) unique nucleic acid tags, each unique nucleic acid tag of each compound includes at least one and preferably two or more catenated different hybridization sequences, wherein the hybridization sequences are capable of binding specifically to complementary immobilized capture nucleic acid sequences, and (b) a chemically reactive reaction moiety that may include a compound precursor, a partially synthesized compound, or completed compound. A nucleic acid tagged compound in which the chemically reactive moiety is a completed-synthesis compound is also referred to as a nucleic acid-tagged compound.

The term “small molecule” refers to a compound having a molecular weight of between 100 and 1000 daltons.

As used herein, the term “combining” refers to placing reagents in a way that allows the reagents to react with one other. The term “combining” includes mixing. In combining three or more reagents, the reagents may be combined in any logical order (e.g., one after the other or all at the same time).

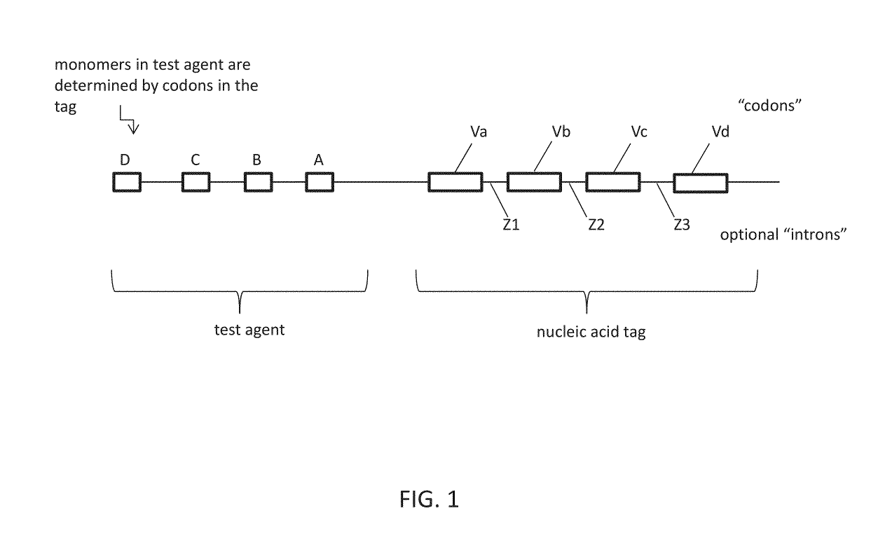

As used herein, the term “nucleic acid-programmed small molecule library” refers to a library of molecules each of which comprises: a) a test agent that is composed of a string of monomers that are covalently attached and b) a nucleic acid tag that encodes and has directed the synthesis of the test agent. In some embodiments, library members may contain a cleavable linker between the test agent and the nucleic acid tag. An example of a member of a nucleic acid-programmed small molecule library is schematically illustrated in FIG. 1. The order of the monomers in the test agent does not need to be the same as the order of the sequences that encode the monomers in the nucleic acid. Nucleic acid-programmed small molecule libraries are described in a variety of publications including: Weisinger et al. (PLoS One 2012 7:e32299), Weisinger et al. (PLoS One. 2012 7:e28056), Wrennet et al. (J Am Chem Soc. 2007 129:13137-43), Wrenn et al. (Annual Rev. Biochem. 2007 76:331-49), Halpin et al (PLoS Biol. 2004 2:E175), Halpin et al. (PLoS Biol. 2004 2:E174), Halpin et al. (PLoS Biol. 2004 2:E173), which are incorporated by reference for a description of the libraries, methods for their construction (which involve a “split and pool” approach) and reagents for the same.

As used herein, the term “test agent” is a polymer of monomers, where the monomers of a test agent may amino acid residues, non-amino acid residues, or a mixture of the two. The monomers may be attached using any suitable linkage.

As used herein, the term “nucleic acid tag that encodes the test agent” refers to a nucleic acid tag that is covalently attached to the test agent in the library. The length of the tag may vary greatly depending on the number of monomers encoded by the tag, the size of the codons and the length of the introns, if used. In some embodiments, the nucleic acid tag is from 100 nt to 300 nt in length and may contain 3-10 or more “codons” (which may be in the range of, e.g., 10-30 nucleotides in length) that are separated by non-coding “introns” (which may be in the range of, e.g., 10-30 nucleotides in length) that are used for tag assembly.

The term “recombining” refers to the formation of chimeras of nucleic acid tags derived from selected members of a library. Chimeras can be formed by PCR amplification, partial digestion, hybridization and primer extension, for example, although other methods are known.

The term “chemoselective functional group” refers to a reactive group that is not already present on the test agents in a library, i.e., an “orthogonal” group. For example, a thiol group (which is reactive with iodoacetamide) is orthogonal if the test agents do not contain any thiol groups. Likewise, the reactive groups used in click chemistry (e.g., azide and alkyne groups) are orthogonalif they are not already present in the test agents in the library.

The term “capture molecule” refers to a molecule that can be used to capture library members that have been modified to contain a chemoselective functional group. Capture agents contain a group that reacts with the chemoselective functional group (e.g., an active ester such as an amino-reactive NHS ester, a thiol-reactive maleimide or iodoacetamide groups, an azide group or an alkyne group, etc). In some embodiments a capture molecule may be bifunctional in that it may also contain a capture moiety, such as a biotin moiety, that can be used to anchor reaction products to a substrate, e.g., beads or the like. In some embodiments, the capture molecule may be directly linked to a substrate without a capture moiety.

As used herein, the term “biotin moiety” refers to an affinity agent that includes biotin or a biotin analogue such as desthiobiotin, oxybiotin, 2′-iminobiotin, diaminobiotin, biotin sulfoxide, biocytin, etc. Biotin moieties bind to streptavidin with an affinity of at least 10⁻⁸M. A biotin affinity agent may also include a linker, e.g., -LC-biotin, -LC-LC-Biotin, -SLC-Biotin or -PEGₙ-Biotin where n is 3-12.

Other definitions may be found in the detailed description.

### DETAILED DESCRIPTION OF EXEMPLARY EMBODIMENTS

As noted above, this disclosure provides method of screening comprising: a) combining a nucleic acid-programmed small molecule library with: an enzyme, and a substrate for the enzyme, wherein each of the members of the library comprises a test agent that is linked to an nucleic acid tag that encodes the test agent and the combining results in transferring a chemoselective functional group from the substrate onto at least some of the members of the library; b) isolating the library members onto which the chemoselective functional group has been covalently transferred; and c) amplifying the nucleic acid tags of the library members isolated in step b) to produce an amplification product. Libraries and kits for performing the method are also provided as are compounds and pharmaceutical compositions thereof.

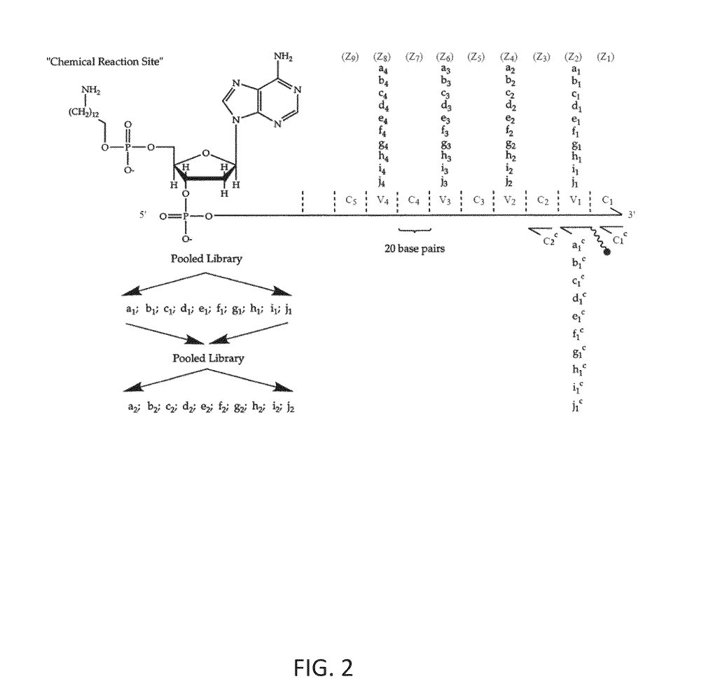

The nucleic acid tags of the library may be composed of 3 to 20 or more regions of different catenated nucleic acid sequences and a chemical reaction site. In the example shown in FIG. 2, some of these regions are denoted C₁ through C₅ and refer to the “constant”, “spacer” or “intron” sequences that may, in certain embodiments, be the same for the nucleic acid tags. The four V regions denoted V₁ through V₄ refer to the “variable” hybridization sequences at the first through fourth positions. In representative embodiments, the V regions and C regions alternate in order from the 3′ end of the nucleic acid tag to the 5′ end of the nucleic acid tag.

The variable hybridization sequences are generally different for each group of sub-population of nucleic acid tags at each position. In the above embodiment, every V region is bordered by two different C regions. As will be appreciated from below, all of the V-region sequences are orthogonal, such that no two V-region sequences cross-hybridize with each other. For example, in an embodiment that comprises nucleic acid tags that include four variable regions and 400 different nucleic acid sequences for each of the four variable regions, there are a total of 1,600 orthogonal nucleic acid hybridization sequences. Such hybridization sequences can be designed according to known methods. For example, where each variable hybridization sequence comprises 20 nucleotides, with a possibility of one of four nucleotides at each position, 20⁴ different sequences are possible. Of the different possible candidates, specific sequences can be elected such that each sequence differs from another sequence by at least 2 to 3, or more, different internal nucleotides.

In general suitable C and V regions comprise from about 10 nucleotides to about 30 nucleotides in length, or more. In certain embodiments, C and V regions comprise from about 11 nucleotides to about 29 nucleotides in length, including from about 12 to about 28, from about 13 to about 27, from about 14 to about 26, from about 14 to about 25, from about 15 to about 24, from about 16 to about 23, from about 17 to about 22, from about 18 to about 21, from about 19 to about 20 nucleotides in length. In representative embodiments, C and V regions comprise about 20 nucleotides in length.

A nucleic acid tag can comprise from about 1 to about 100 or more different V regions (hybridization sequences), including about 200, about 300, about 500, or more different V regions. In representative embodiments, a nucleic acid tag comprises from about 1 to about 50 different V regions, including about 2 to about 48, about 3 to about 46, about 4 to about 44, about 5 to about 42, about 6 to about 40, about 7 to about 38, about 8 to about 36, about 9 to about 34, about 10 to about 32, about 11 to about 30, about 12 to about 29, about 13 to about 28, about 14 to about 27, about 15 to about 26, about 16 to about 25, about 17 to about 24, about 18 to about 23, about 19 to about 22, about 20 to about 21 different V regions.

A nucleic acid tag can comprise from about 1 to about 100 or more different C regions (constant sequences), including about 200, about 300, about 500, or more different C regions. In representative embodiments, a nucleic acid tag comprises from about 1 to about 50 different C regions, including about 2 to about 48, about 3 to about 46, about 4 to about 44, about 5 to about 42, about 6 to about 40, about 7 to about 38, about 8 to about 36, about 9 to about 34, about 10 to about 32, about 11 to about 30, about 12 to about 29, about 13 to about 28, about 14 to about 27, about 15 to about 26, about 16 to about 25, about 17 to about 24, about 18 to about 23, about 19 to about 22, about 20 to about 21 different C regions.

As noted above, a population of nucleic acid tags is degenerate, i.e., almost all of the nucleic acid tags differ from one another in nucleotide sequence. The nucleotide differences between different nucleic acid tags reside entirely in the hybridization sequences (V regions). For example, an initial population of nucleic acid tags can comprise of 400 first sub-populations of nucleic acid tags based on the particular sequence of V₁ of each sub-population. As such, the V₁ region of each sub-population comprises of any one of 400 different 20 base-pair hybridization sequences. Separation of such a population of nucleic acid tags based on V₁ would result in 400 different sub-populations of nucleic acid tags. Likewise, the same initial population of nucleic acid tags can also comprise of 400 second sub-populations of nucleic acid tags based on the particular sequence of V₂ of each sub-population, wherein the second sub-populations are different than the first sub-populations.

In the exemplary population of nucleic acid tags demonstrated in FIG. 2, the first few of the first hybridization sequences are denoted as a₁, b₁, c₁ . . . j₁, in the V₁ region of the different nucleic acid tags. Likewise, the first few of the second hybridization sequences are denoted as a₂, b₂, c₂ . . . j₂, in the V₂ region of the different nucleic acid tags. The first few of the third hybridization sequences are denoted as a₃, b₃, c₃ . . . j₃, in the V₃, etc.

In certain embodiments, the nucleic acid tags share the same twenty base-pair sequence for designated spacer regions while having a different twenty base-pair sequence between different spacer regions. For example, the nucleic acid tags comprise the same C₁ spacer region, the same C₂ spacer region, and the same C₃ spacer region, wherein C₁, C₂, and C₃ are different from one another.

Thus each 180 nucleotide long nucleic acid tag may be composed of an ordered assembly of 9 different twenty base-pair regions comprising the 4 variable regions (a₁, b₁, c₁ . . . d₅, e₅, f₅, . . . h₁₀, i₁₀, j₁₀) and the 5 spacer regions (z₁ . . . z₁₁) in alternating order. The twenty base-pair regions have the following properties: (i) micromolar concentrations of all the region sequences hybridize to their complementary DNA sequences efficiently in solution at a specified temperature designated Tm, and (ii) the region sequences are orthogonal to each other with respect to hybridization, meaning that none of the region sequences cross-hybridizes efficiently with another of the region sequences, or with the complement to any of the other region sequences, at the temperature Tm.

The degenerate nucleic acid tags can be assembled from their constituent building blocks by the primerless PCR assembly method described by Stemmer et al., Gene 164(1):49-53 (1995), and the tags may be used to direct that synthesis of the test agents using the methods described by Weisinger et al. (PLoS One 2012 7:e32299), Weisinger et al. (PLoS One. 2012 7:e28056), Wrennet et al. (J Am Chem Soc. 2007 129:13137-43), Wrenn et al. (Annual Rev. Biochem. 2007 76:331-49), Halpin et al (PLoS Biol. 2004 2:E175), Halpin et al. (PLoS Biol. 2004 2:E174), Halpin et al. (PLoS Biol. 2004 2:E173).

As noted above the nucleic acid tags further comprise a chemical reaction site at any site, including the 3′ terminus, the 5′ terminus, or any other position on the nucleic acid tag. In some embodiments, the chemical reaction site can be added by modifying the 5′ alcohol of the 5′ base of the nucleic acid tag with a commercially available reagent which introduces a phosphate group tethered to a linear spacer, e.g., a 12-carbon chain terminated with a primary amine group (e.g., as available from Glen Research, or numerous other reagents which are available for introducing thiols or other chemical reaction sites into synthetic DNA).

The chemical reaction site is the site at which the particular compound is synthesized and may be dictated by the order of V region sequences of the nucleic acid tag. An exemplary chemical reaction site is a primary amine. Many different types of chemical reaction sites in addition to primary amines can be introduced at any site, including the 3′ terminus, the 5′ terminus, or any other position on the nucleic acid tag. Exemplary chemical reaction sites include, but are not limited to, chemical components capable of forming amide, ester, urea, urethane, carbon-carbonyl bonds, carbon-nitrogen bonds, carbon-carbon single bonds, olefin bonds, thioether bonds, and disulfide bonds. In the case of enzymatic synthesis, co-factors may be supplied as are required for effective catalysis. Such co-factors are known to those of skill in the art. An exemplary cofactor is the phosphopantetheinyl group useful for polyketide synthesis. The test agent may be composed of any suitable monomer, e.g., amino acids and analogs thereof and/or non-amino acid building blocks (see, e.g., US20090264300, which is incorporated by reference for disclosure of non-amino acid building blocks). A test agent may contain, for example, 3, 4, 5, 6, 7, 8, 9 10 or more monomers. The monomers can be joined together using any suitable chemistry, e.g., amine acylation, reductive alkylation, aromatic reduction, aromatic acylation, aromatic cyclization, aryl-aryl coupling, [3+2]cycloaddition, Mitsunobu reaction, nucleophilic aromatic substitution, sulfonylation, aromatic halide displacement, Michael addition, Wittig reaction, Knoevenagel condensation, reductive amination, Heck reaction, Stille reaction, Suzuki reaction, Aldol condensation, Claisen condensation, amino acid coupling, amide bond formation, acetal formation, Diels-Alder reaction, [2+2]cycloaddition, enamine formation, esterification, Friedel Crafts reaction, glycosylation, Grignard reaction, Horner-Emmons reaction, hydrolysis, imine formation, metathesis reaction, nucleophilic substitution, oxidation, Pictet-Spengler reaction, Sonogashira reaction, thiazolidine formation, thiourea formation and urea formation.

The nucleic acid-programmed small molecule library may have a complexity of at least 10⁶, at least 10⁷, at least 10⁹, at least 10¹⁰, at least 10¹¹, at least 10¹², etc.

The enzyme used in the method may be any enzyme that can post-translationally modify another biological entity, e.g. a protein, in a sequence-selective manner. These enzymes include, but are not limited to, kinases, isoprenylases, acylases, oxidases, glycosylases, amidases, methylases and a variety of enzymes that catalyze myristoylation (attachment of myristate, a C14 saturated acid), palmitoylation (attachment of palmitate, a C16 saturated acid), (isoprenylation or prenylation (the addition of an isoprenoid group (e.g. farnesol and geranylgeraniol), glypiation (glycosylphosphatidylinositol (GPI) anchor formation via an amide bond to C-terminal tail), cofactor addition (e.g., the attachment of a lipoate, a flavin moiety (FMN or FAD), heme C, the addition of a 4′-phosphopantetheinyl moiety from coenzyme A, retinylidene), diphthamide formation, ethanolamine phosphoglycerol, hypusine formation, as well as enzymes that catalyze acylation (e.g. O-acylation, N-acylation, or S-acylation), acetylation (either at the N-terminus or at lysine residues), formylation, alkylation (i.e., the addition of an alkyl group, e.g. methyl, ethyl), methylation (e.g., at a lysine or arginine residue), amide bond formation, amidation at C-terminus, amino acid addition, arginylation, polyglutamylation, polyglycylation, butyrylation, gamma-carboxylation, glycosylation (the addition of a glycosyl group to either arginine, asparagine, cysteine, hydroxylysine, serine, threonine, tyrosine, or tryptophan resulting in a glycoprotein), polysialylation, malonylation, iodination (e.g. of thyroglobulin), nucleotide addition such as ADP-ribosylation, phosphate ester (O-linked) or phosphoramidate (N-linked) formation, phosphorylation, the addition of a phosphate group (usually to serine, threonine, and tyrosine (O-linked), or histidine (N-linked)), adenylylation (the addition of an adenylyl moiety, usually to tyrosine (O-linked), or histidine and lysine (N-linked)), propionylation, pyroglutamate formation, S-glutathionylation, S-nitrosylation, succinylation addition of a succinyl group to lysine, sulfation, or the addition of a sulfate group to a tyrosine or selenoylation.

In certain embodiments, the enzyme used may be a protein kinase (EC 2.7.11 or 2.7.12) and in other embodiments may an Abl, ALK, AMPK, Arg, Aurora-A, Axl, Blk, Bmx, BTK, CaMKII, CaMKIV, CDK1/cyclinB, CDK2/cyclinA, CDK2/cyclinE, CDK3/cyclinE, CDK5/p35, CDK6/cyclinD3, CDK7/cyclinH/MAT1, CHK1, CHK2, CK1δ, CK2, c-RAF, CSK, cSRC, EGFR, EphB2, EphB4, Fes, FGFR3, Flt3, Fms, Fyn, GSK3α, GSK3β, IGF-1R, IKKα, IKKβ, IR, JNK1α1, JNK2α2, JNK3, Lck, Lyn, MAPK1, MAPK2, MAPKAP-K2, MEK1, Met, MKK4, MKK6, MKK7.beta., MSK1, MST2, NEK2, p70S6K, PAK2, PAR-1α, PDGFRα, PDGFRβ, PDK1, PKA, PKBα, PKBβ, PKBγ, PKCα, PKCβII, PKCγ, PKCδ, PKCε, PKCmu, PKCtheta, PKCzeta, PKD2, PRAK, PRK2, ROCK-II, ROCK-II, Ros, Rsk1, Rsk2, Rsk3, SAPK2a, SAPK2b, SAPK3, SAPK4, SGK, Syk, Tie2, TrkB, Yes & ZAP-70 kinase. In some embodiments, the kinase used may have an amino acid sequence that is at least 80% identical to (e.g., at least 90% identical to at least 95% identical to at least 98% identical to) naturally occurring kinase.

The substrate used in the method may be a native substrate for the enzyme (if the substrate already has a chemoselective functional group) or a modified form of the native substrate that has a chemoselective functional group. For example, the substrate may contain a thiol group, an amine group, a carboxyl, an azide or an alkyne group that get transferred to at least some of the members of the library by the enzyme. In some embodiments, the substrate is alpha-thio-ATP, although other native substrates can be modified in a similar way to transfer thiol or another group to the library. The substrate used in the method may be chosen to be compatible with the enzyme and the test agents of the library. Chemoselective functional groups of interest include, but are not limited, to, thiol, thiophosphate, iodoacetyl groups, maleimide, azido, alkynyl (e.g., a cyclooctyne group), phosphine groups, Click chemistry groups, groups for Staudinger ligation, and the like. A thiol or thiophosphate group may be compatible with an iodoacetyl group and/or a maleimide group. Azido and alkynyl groups may be conjugated via a Click chemistry. Any convenient cycloaddition chemistry, including Click chemistries or Staudinger ligation chemistries may be utilized.

As noted above, the library is combined under conditions in which the enzyme covalently transfers a chemoselective functional group from said substrate to one or more library members. Such conditions may be readily adapted from what is already known in the art.

After the library, the enzyme and substrate have been incubated for a defined period of time (e.g., from 5 minutes to 24 hours), the library members onto which the chemoselective functional group have been covalently transferred are isolated from the other library members. In some embodiments, the chemoselective functional group is reacted with a moiety to provide a covalent linkage, wherein the moiety is bound to a solid support, or contains a capture moiety (e.g., a biotin moiety) that can be bound to a solid-support (e.g., one that contains streptavidin, for example).

Next, after the library members onto which the chemoselective functional group have been covalently transferred have been isolated, the nucleic acid tags of the isolated library members can be amplified to produce an amplification product that in certain embodiments may be used to direct the synthesis of a further small molecule library that is screened in the same way. In some embodiments, the amplifying may be done by PCR using primers that hybridize to or are the same as universal primer sequences that are ends all of the nucleic acid tags in the library. In some embodiments, the amplifying may comprise mutagenizing and/or recombining the nucleic acid tags of the isolated library members with one another to produce new nucleic acid tags thereby permitting “evolution” of the isolated test agents. More specifically, genetic recombination between the nucleic acid tags that encode selected test agents may be carried out in vitro by mutagenesis or random fragmentation of the nucleic acid tag sequence, followed by the generation of related nucleic acid sequences (“gene shuffling”, Stemmer, Nature, 370: 389391 (1994); U.S. Pat. No. 5,811,238). In some embodiments, a unique restriction site is introduced into each specific hybridization sequence. By way of example, partial digestion of a library may be done using a plurality of different restriction enzymes, followed by a primerless PCR reassembly reaction. By analogy to gene shuffling for protein synthesis (Crameri, et al., Nature 391 (6664): 288-291 (1998)), the ability to carry out genetic recombination of compound libraries vastly increases the efficiency with which the diversity in the compound libraries can be explored and optimized. The recombination step yields a population of variant nucleic acid sequences, capable of directing the synthesis of structurally-related, and/or functionally-related molecules, and/or variants thereof to create compounds having one or more desired activities.

In some embodiments, the method comprises making a second nucleic acid-programmed small molecule library using the amplification product (which may or may not have been shuffled). In some embodiments, the method further comprises rescreening the second nucleic acid-programmed small molecule library (i.e., by combining the second nucleic acid-programmed small molecule library with the same enzyme and a suitable substrate for the enzyme (which may or may not be the same substrate as used earlier in the protocol), where the enzyme covalently transfers the chemoselective functional group to at least one library member. As with earlier in the protocol, the re-screening method may comprise isolating the library members onto which the chemoselective functional group has been covalently transferred and, as before, amplifying the oligonucleotide tags of the isolated library members.

These steps (i.e., library synthesis, test agent selection, and amplification of the selected sequences, with optional shuffling) may be successively repeated one or more times (e.g., 2, 3, 4, 5, or 6 or more times) to produce a final amplification product. This final amplification product may be sequenced and decoded to identify a test agent onto which the chemoselective functional group has been attached. In some embodiments, the sequences may be aligned to provide a consensus sequence for the test agent.

As would be apparent, the primers used for amplification may be compatible with use in a next generation sequencing platform, e.g., Illumina's reversible terminator method, Roche's pyrosequencing method (454), Life Technologies' sequencing by ligation (the SOLiD platform) or Life Technologies' Ion Torrent platform. Examples of such methods are described in the following references: Margulies et al. (Nature 2005 437: 376-80); Ronaghi et al. (Analytical Biochemistry 1996 242: 84-9); Shendure (Science 2005 309: 1728); Imelfort et al. (Brief Bioinform. 2009 10:609-18); Fox et al. (Methods Mol Biol. 2009; 553:79-108); Appleby et al. (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps. In certain embodiments, the primers may provide two sets of primer binding sites, one for amplifying the products and the other for sequencing the resultant product. In other embodiments, the sequencing primer sites may be added by amplifying the final PCR products with tailed primers, where the tails of those primers provide primer binding sites.

After identifying a test agent onto which the chemoselective functional group has been attached, the test agent may be made again and tested without the nucleic acid tag to determine if it can act as a substrate for the enzyme. In some embodiments, the identified test agent may act as an inhibitor of the enzyme, or it may be used as an affinity tag for the enzyme.

In alternative embodiments, the modified test agents may be isolated using an antibody that binds to the transferred group. For example, if the enzyme is a ubiquitin tagging enzyme, such as a ubiquitin-activating enzyme (E1), a ubiquitin-conjugating enzyme (E2), or a ubiquitin ligase (E3), the ubiquitin tagged test agents may be isolated using an antibody that binds to ubiquitin.

Also provided herein is a nucleic acid-programmed small molecule library that can be split into various portions (e.g., 2, 3, 4, 5, or 6 or more portions), where the portions are essentially identical to one another except for a tag sequence that allows the portions to be separated from one another. This library comprises at least: a) a first set of members of a nucleic acid-programmed small molecule library; and b) a second set of members of a nucleic acid-programmed small molecule library, wherein the first and second sets of members of the library are essentially identical except for a tag sequence that allows the first and second sets of members to be separated from one another by hybridization. The tag sequences, which are part of the nucleic acid tag, do not hybridize to one another or their complements, and may be 10-30 nt in length.

This library can be split into portions by hybridization, and the portions can be subjected to different conditions. In some embodiments, the portions can be treated with different enzymes and in other embodiments one of the portions can be used as a control (e.g., to provide a “mock” treatment controls) for another of the portions. In some embodiments, the method comprises: a) making a nucleic acid-programmed small molecule library that comprises at least a first set of members and a second set of members, wherein the first set of members and the second set of members are essentially identical except for a sequence tag that allows the first and second sets of members to be separated from one another by hybridization; and b) separating the first and second sets from one another by hybridization to the sequence tag. In some embodiments the method further comprise screening the first set of library members under a first set of conditions to obtain first results (e.g., using the method described above to obtain a first set of library members onto which a chemoselective functional group has been transferred by an enzyme) and also screening the second set of library members under a second set of conditions to obtain second results (e.g., using the method described above to obtain a first set of library members onto which a chemoselective functional group has been transferred by another enzyme or in the absence of the enzyme). The results from one assay can be compared to the results of the other assay, e.g., to evaluate the confidence of any of the results.

In some embodiments, a method of measuring gene enrichment is provided. The method comprises: a) making a nucleic acid-programmed small molecule library that comprises at least a first set of members and a second set of members, wherein the first set of members and the second set of members are essentially identical except for a sequence tag that allows the first and second sets of members to be separated from one another by hybridization; b) screening the first set of library members under a first set of conditions to obtain first results (e.g., using the method described above to obtain a first set of library members onto which a chemoselective functional group has been transferred by an enzyme) and not screening the second set of library member; (c) separating the first and second sets from one another by hybridization to the sequence tag; and (d) calculating the ratio of a gene's fractional abundance in the first set of library members relative to its fractional abundance in the second population.

### Compounds and Compositions

A compound comprising at least one amino acid sequence selected from the group consisting of: RRSFL, RRSFV, RRASL, RRFSV, RRMSV, RRMTV, RMSF, RRSF and RRMS is provided. The peptide may be of any length, e.g., 4 amino acids, 5 amino acids, 6 amino acids, 7 amino acids, 8 amino acids, 9 amino acids, at least 10 amino acids, or at least 20 amino acids up to 50 amino acids or more in length. A pharmaceutical composition comprising the compound and a pharmaceutically acceptable excipient is also provided. Pharmaceutically acceptable excipients are well know to those of skill in the art and may be found, inter alia, in compendiums such as Remington: The Science and Practice of Pharmacy, 22 Edition, Lloyd V. Allen, Jr, Ed.

### Kits

Also provided herein are kits for practicing the subject methods, as described above. In certain embodiments, a kit may include a) a nucleic acid-programmed molecule library, wherein each library member comprises a test agent that is linked to an nucleic acid tag that encodes the test agent; b) an enzyme; and c) a substrate for the enzyme, wherein the enzyme covalently transfers a chemoselective functional group from said substrate to one or more library members. The kit may further comprise a capture molecule that reacts with the chemoselective functional group, as described above. In certain embodiments, the capture molecule may contain a biotin moiety. A subject kit may also include one or more other reagents for preparing or processing a library, including PCR primers, an affinity support, etc.

In addition to above-mentioned components, the subject kits typically further include instructions for using the components of the kit to practice the subject methods, i.e., to synthesize a combinatorial library using the subject device and/or screening a combinatorial library according to the subject methods. The instructions for practicing the subject methods may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control analyte mixtures, e.g., two or more control samples for use in testing the kit. Although the foregoing embodiments have been described in some detail by way of illustration and example for purposes of clarity of understanding, it is readily apparent to those of ordinary skill in the art in light of the above teachings that certain changes and modifications can be made thereto without departing from the spirit or scope of the appended claims.

### EXAMPLES

Aspects of the present teachings can be further understood in light of the following example, which should not be construed as limiting the scope of the present teachings in any way.

### Methods

### Reagents

Solvents and general chemistry reagents were purchased from VWR International (West Chester, Pa.), Fisher Scientific (Hampton, N.H.), or Sigma-Aldrich (St. Louis, Mo.). Fmoc protected amino acids were from Novabiochem (La Jolla, Calif.) or Chem-Impex (Wood Dale, Ill.). Oligonucleotides were purchased from Bioneer (Alameda, Calif.) and the Stanford PAN Facility (Stanford, Calif.). The catalytic subunit of murine cAMP-activated protein kinase A was from New England Biolabs (NEB, Ipswich, Mass.) (catalog # P6000L) and peptides were obtained from AnaSpec (Fremont, Calif.).

### DNA-Programmed Combinatorial Chemistry

Programmed library synthesis was carried out by a series of DNA-directed library splitting steps followed by chemical coupling steps. For the splitting steps, the ssDNA library was hybridized to an anticodon array overnight at 37° C. using a mesofluidic hybridization pump (see, e.g., Weisinger et al. (2012) PLoS ONE, 7, e28056). The anticodon array with bound DNA was then mounted into a plate adapter device which uses rubber gaskets to form an isolated liquid channel above and below each array feature. The adapter device was placed on top of a 384-well polypropylene filterplate loaded with 5 μl aliquots of DEAE sepharose resin. DNA was eluted from each feature of the anticodon array onto the DEAE resin in the corresponding well of the filterplate by application of a denaturing buffer (10 mM NaOH with 1 mM EDTA and 0.005% Triton X-100) followed by gentle centrifugation of the stack (140×g for 1 minute). The DEAE resin in the filter plate was then washed three times with 85 μl H₂O and three times with 85 μl dry methanol. Peptide couplings were performed using EDC and HOAt in methanol and DMF (see, e.g., Halpin et al., (2004) PLoS Biology, 2, E174). Following the synthetic steps, the filterplate was placed on top of a 384-well polypropylene microtiter plate, and DNA was eluted from the DEAE resin by application of a high salt buffer (1.5 M NaCl, 50 mM NaOH, 1 mM EDTA, and 0.005% Triton X-100) followed by gentle centrifugation. The eluted DNA was pooled, concentrated and buffer exchanged into hybridization buffer using a centrifugal filter device with a 10,000 Da molecular weight cut-off (GE Healthcare). BSA and tRNA were added to 0.5 mg/ml each. The sample was diluted to 3 ml with hybridization buffer and applied to another anticodon array. Following the final step of the chemical synthesis, the eluted DNA was split by hybridization for 3 hours at 37° C. to a pair of anticodon columns derivatized with the VE₀₀₁ and VE₀₀₂ anticodon sequences. The two halves of the library were concentrated with n-butanol extractions, precipitated with isopropanol, and subjected to kinase-substrate/mock selections respectively.

### Selection for Protein Kinase A Substrates

Translated libraries were incubated in PKA buffer (NEB) overnight at 30° C. with 10,000 units of protein kinase A and 1 mM ATPγS. For the selection between the third and fourth generations, the incubation was shortened to 1 hour at 30° C. Mock selections were performed identically, with the enzyme replaced by a 50% glycerol solution. After incubation, the crude reactions were diluted 1.5-fold and adjusted to 100 mM NaOAc pH 5.2 and 25% dimethyl formamide. EZ-Link iodoacetyl-LC-biotin (Pierce Thermo Fisher Scientific, catalog #21333) was added to 600 μM. After 3 hours in the dark at 25° C., the enzyme-treated library was cleaned up by extraction with phenol: chloroform and n-butanol, and then precipitation with isopropanol. The library was resuspended in 27.5 μl bind buffer (10 mM Tris pH 7.4, 1 mM EDTA, 1 M NaCl), and 1.25 mg/ml tRNA and 0.25 mg/ml BSA were added. The library was then incubated with 15 μl of μMACS streptavidin microbeads (MiltenyiBiotec, 130-074-101) overnight at room temperature. The microbeads were purified and washed over MACS columns according to the manufacturer's instructions, and the purification process was repeated a second time. DNA was amplified directly off of the streptavidin beads by PCR.

### Illumina Sequencing

25 pmol of amplified DNA was used as a template in a PCR reaction that appended Illumina adaptor sequences. The PCR product was quantified on an Agilent 2100 Bioanalyzer, and a 10 nM solution was submitted to the Stanford Functional Genomics Facility for sequencing using custom primers. Paired-end 150-bp reads were obtained on a MiSeqIllumina Sequencer.

### Data Analysis

The sequencing data were processed using scripts written in either AWK or MATLAB. First, a string search on the fastqMiSeq files was used to locate constant-region sequences (Zₐ-Z_{f}), and the 20 base-pair blocks adjacent to each constant region were excised and saved. The forward reads gave sequences for the first four codons (A-D), and the reverse reads gave reverse-complement sequences for the last five codons (B-E). Redundant forward and reverse reads were obtained for the three central codons (B-D). The 20mer blocks were converted into codon numbers using a direct string comparison to each of the 384 possible codon sequences. In order for a codon number assignment to be made, at least 18 bases had to match between the observed 20mer and the reference codon sequence. Paired reads that gave contradictory assignment at codons B-D were discarded. The raw list of codon sequences was sorted, and the reads were summed, to generate a non-redundant list of codon sequences and the number of times that each sequence was observed in the data. The codon-sequence data were then split into a kinase-selected block and a mock-selected block based on the identity of the E codon. The codon frequencies at each coding position in the mock-selected library were calculated, and the fractional abundance of each four-number codon sequence in the mock library was approximated as the product of the frequencies of its constituent codons.

### K_{cat}/Kₘ Measurements

Candidate peptides with a C-terminal amide were synthesized by a commercial vendor. Phosphorylation of the peptides by protein kinase A was monitored based on HPLC retention time. Initial rates under non-saturating conditions were measured at multiple enzyme and peptide concentrations, and the data were used to fit a second order rate constant.

### Results

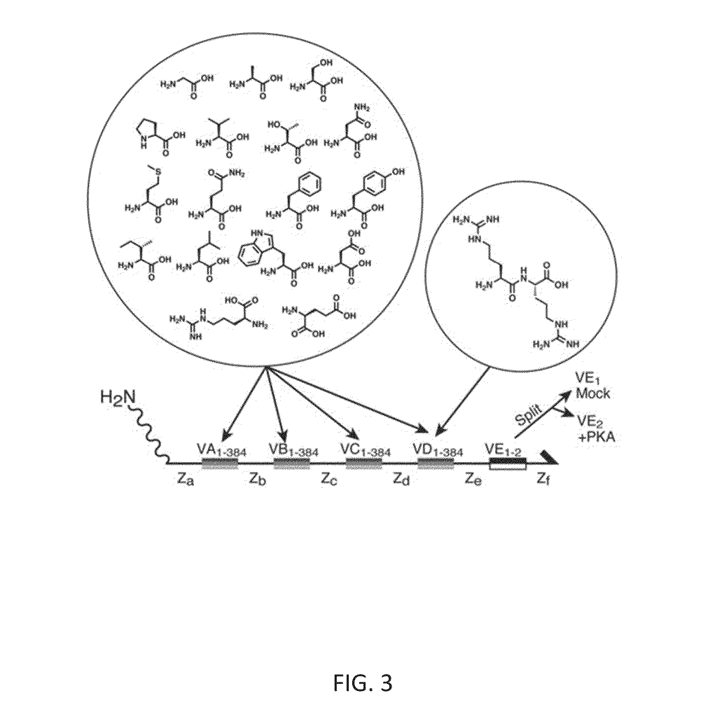

DNA-programming of an n-step chemical synthesis in microplate format can accommodate a library complexity of 384ⁿ. Our experiment used this coding capacity redundantly. We worked with a peptide library that was assembled in four coupling steps, using seventeen different amino acids as building blocks in the first three steps, and eighteen in the fourth step (FIG. 3). The eighteenth building block in the final coupling step was an arginine dipeptide, so a portion of the peptides consisted of pentamers. Six DNA codons were assigned to each of the amino acids. Ten billion different DNA genes programmed the synthesis of a peptide, but the peptides only covered 110,808 different amino-acid sequences. The library was subjected to a selection for protein kinase A substrates based on a phosphopeptide enrichment scheme. The DNA-peptide conjugate library was first incubated with protein kinase A and ATP gamma-S, and then treated with biotin-iodoacetamide. This procedure covalently links biotin to peptides that were thiophosphorylated by the kinase. The biotinylated DNA-peptide conjugates were then isolated on streptavidin coated paramagnetic beads. Initial substrate enrichment using mock selections with purified substrate and non-substrate conjugates was quantified. These test selections produced substrate enrichments of 1000-fold.

In experiments with full peptide libraries, an internal-control selection was performed in parallel with the kinase selection. The control selection was designed to reveal gene enrichment caused by factors unrelated to the proficiency of the encoded peptide substrates. It was identical to the substrate selection except that the kinase enzyme was omitted. The control DNA-peptide conjugates were distinguished from the kinase-treated DNA-peptide conjugates by a barcode inserted into the DNA genes (FIG. 3). The two types of genes were pooled and chemically translated together. They were then separated from each other after library synthesis by hybridization of the bar-code sequences to two different oligonucleotide affinity resins. The DNA-peptide conjugate library was evolved over four generations (the initial gene population was designated as generation zero). Each round consisted of a chemical translation step, the application of selective pressure favoring kinase substrates, and then the amplification and diversification of the enriched genes. The gene populations of the second, third and fourth generations (the grandchildren, great-grandchildren and great-great grandchildren of the initial population) were sequenced and analyzed. Gene enrichment was calculated as the ratio of a gene's fractional abundance in the kinase-treated population relative to its apparent fractional abundance in the control population. By the fourth generation, 999 of the 1000 most abundant genes coded for a serine or threonine as well as an N-terminal arginine (versus 3 expected by chance). This predominance of putative substrate motifs was absent in the ancestor population: only 6 of the 1000 most abundant genes in the initial library met the same criteria. Between the second and third generations, many substrate-encoding genes became sufficiently enriched to appear on average at least twice in ˜3 million sequencing reads. This threshold corresponds to a 15,000-fold cumulative enrichment on a per-gene basis. Interestingly, substrate-encoding genes were enriched by only 10-50 fold per selection step, not 1000-fold as observed in mock selections. To identify the most highly enriched peptide in the fourth generation library, the reads for the 6⁴ (1296) different genes corresponding to each single peptide sequence were summed, and then peptide enrichment based on the summed reads were calculated. The most highly enriched peptide had the sequence RRSFL.

The dominant sequence motifs in the fourth generation data set were RRB[S/T]B and RRSFB, where B denotes a non-polar residue (see Table 1 below). The first motif corresponds to a known consensus sequence for PKA substrates. The second motif, however, was unexpected. The registration between the arginine and serine residues is unusual among known PKA substrates. To evaluate identified sequence binding motifs, k_{cat}/K_{M} values were measured for eight peptides with diverse enrichment rankings (see Table 1 below). Peptides in the alternate class were bona fide PKA substrates, but their phosphorylation was less efficient than for peptides in the canonical class. Notably, two of the evolved pentapeptide mini-substrates were phosphorylated more rapidly than the Kemptide heptapeptide, which has been considered an optimal PKA substrate for thirty years. One tetrapeptide weighing only 565 daltons had a k_{cat}/K_{M} value only 20-fold lower than that of Kemptide. A gross correlation between substrate proficiency and gene enrichment was observed, but this correlation did not hold at a granular level.

(SEQ ID NO: 1)
RRSFL,
(SEQ ID NO: 2)
RRSFV,
(SEQ ID NO: 3)
RRASL,
(SEQ ID NO: 4)
RRFSV,
(SEQ ID NO: 5)
RRMSV,
(SEQ ID NO: 6)
RRMTV,
(SEQ ID NO: 7)
RMSF,
(SEQ ID NO: 8)
RRSF
and
(SEQ ID NO: 9)
RRMS

TABLE 1
k_{cat}/K_{M} values for resynthesized peptides.

| Peptide^{[a]} | Rank^{[b]} | LTFE^{[c]} | Rel. k_{cat}/Kₘ^{[d]} |
|---|---|---|---|
| RRSFV | 7 | 11.1 | 0.16 |
| (SEQ ID NO: 2) | | | |
| RRASL | 20 | 10.6 | 0.27 |
| (SEQ ID NO: 3) | | | |
| RRFSV | 33 | 10.2 | 4.1 |
| (SEQ ID NO: 4) | | | |
| RRMSV | 51 | 9.5 | 2.2 |
| (SEQ ID NO: 5) | | | |
| RRMTV | 95 | 8.4 | 0.031 |
| (SEQ ID NO: 6) | | | |
| RMSF | 147 | 7.6 | 0.0028 |
| (SEQ ID NO: 7) | | | |
| RRSF | 253 | 6.3 | 0.050 |
| (SEQ ID NO: 8) | | | |
| RRMS | 1846 | 3.0 | 0.028 |
| (SEQ ID NO: 9) | | | |
| LRRASLG^{[e]} | — | — | 1 |
| (SEQ ID NO: 10) | | | |

^{[a]}Peptides were synthesized as C-terminal amides.
^{[b]}Rank: Position on list of peptides ranked by fold enrichment, calculated by summing reads over the 1296 genes encoding each peptide.
^{[c]}LTFE: In (total fold enrichment) after four rounds.
^{[d]}k_{cat}/Kₘ measurements are relative to Kemptide.
^{[e]}Kemptide peptide is present as a C-terminal carboxylate.

Our pilot experiment shows how directed evolution behaves with a fully complex chemical library, meaning a library based on 384 distinct chemical building blocks with one codon per building block. To model how accurately the evolution process can pinpoint the fittest molecules our data was analyzed as though the six codons associated with each amino acid actually represented six “different” amino acids. For example, the six leucine codons would correspond to “leucineA”, “leucineB” and so forth. By splitting each amino acid into six separate entities, the 110,808 original peptide sequences are split into 143,607,168 different virtual peptide sequences. Each of the virtual peptide sequences was encoded by a single gene. The hit molecules in the library were modeled as the set of 1296 virtual RRSFV (SEQ ID NO:2) peptides. Any of the 1296 genes encoding a virtual version of RRSFV (SEQ ID NO:2) was scored as a hit in the top fitness bracket, and all other genes were scored as being sub-optimal.

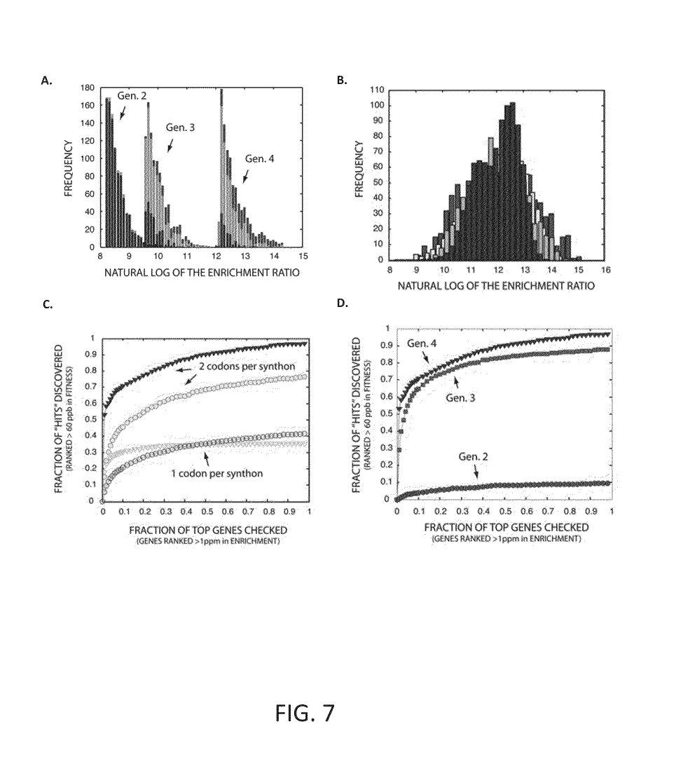

How the enrichment of different hit genes compared with one another was examined. If the properties of the peptide gene product were the sole factor determining enrichment, then all of the hit genes should have been enriched to the same extent. The distribution of log-enrichment in the fourth generation for the genes encoding RRSFV (SEQ ID NO:2) is plotted in FIG. 7B. Ninety-five percent of the measured enrichments covered a range from 28,000-fold to one million-fold, and the median enrichment was 175,000-fold. Poisson noise accounts for one third of the variance in the distribution. The remaining variance indicates that the DNA sequence of the genes influenced enrichment, a phenomenon termed genetic noise. During the transmission of genes over a single generation, the two-sigma variation in log-enrichment due to genetic noise was ˜ 1/9 of the median log-enrichment.

How genetic noise influenced the ranking of RRSFV(SEQ ID NO:2)-encoding genes relative to all other genes was next examined. This is an important question because accurate ranking impacts the utility and efficiency of directed chemical evolution. In particular, screening candidate molecules at the top of a gene enrichment list is the time and resource-limiting step of the process. It requires that compounds be resynthesized individually and then tested for function. In a bad case, one out of every ten molecules might be a hit (a 90% false discovery rate); in the ideal case, all of the molecules would be hits (a 0% false discovery rate). In the absence of genetic noise, the RRSFV(SEQ ID NO:2)-encoding genes should have been stacked at the very top of the enrichment list. In reality, only one half of them were present in the top 20 thousand genes, which is the top 1 part per million of all genes. This half, however, was disproportionately represented at the top. If one had made and tested the encoded peptides descending from the top of the ranked gene list until hitting a 90% false discovery rate, 505 of the 1296 RRSFV(SEQ ID NO:2)-encoding genes would have been discovered.

Understanding the dependence of hit detection on experimental design informs future experiments. Accordingly, how the accuracy of small-molecule ranking depended on three parameters: the number of generations over which the population was evolved, the sequencing depth, and the nature of the genetic code was determined. As a measure of accuracy for this analysis, the number of hit molecules that would have been discovered in the ranked gene list before a 90% false discovery threshold was reached was used. FIG. 7D shows the location of hit genes in a ranked list for second through fourth generations. With a cutoff of a 90% false discovery rate, 0, 207 and 505 of the 1296 hit genes would have been found in the three respective generations. The specificity and sensitivity of hit detection increases dramatically in subsequent generations. To estimate the effect of read depth on ranking accuracy, rank predictions from increasingly small subsets of our sequencing data were compared. The average number of reads per unique gene in the library was varied between 1.5×10⁻⁵ and 1.5×10⁻⁴. The fraction of hits discovered below the 90% false discovery threshold increased from 15% to 43% with a ten-fold increase in number of reads. More generally, the fraction of discovered hits increased roughly as the square root of the number of reads. This type of square-root relationship is characteristic of a signal that is rising above stochastic noise.

The structure of the genetic code also influences how gene products are ranked. One strategy to improve ranking is to use a redundant genetic code, with more than one codon per amino acid (similar to the natural genetic code). This allows enrichment to be averaged over sets of genes that encode the same product, and presumably should reduce the spurious influence of gene DNA sequence on peptide enrichment. Accordingly, a case in which two codons programmed each chemical building block was modeled. The six codons specifying each amino acid were broken into three pairs of two codons. The two codons in each pair were treated as though they coded for the same amino acid, whereas the three separate pairs were treated as though they coded for three different amino acids. For example, the six codons specifying leucine were broken down into a pair that specified “leucine A”, a pair that specified “leucine B”, and a pair that specified “leucine C”, where the A, B and C leucines are viewed as distinct. By splitting each of the amino acids into three, the 110,808 original tetrapeptide sequences are split into 8,975,448 different virtual peptide sequences. Each virtual peptide sequence is encoded by 2⁴=16 different genes. The groups of sixteen related single genes were pooled into gene sets, and the number of sequence reads for each gene set were determined by summing the reads of all its members. Gene-set enrichment was calculated as the ratio of a gene set's fractional gene abundance in the kinase-treated population relative to its apparent fractional abundance in the control population. In order to keep the number of reads per gene product constant relative to the one codon per amino acid case, we used only 1/16th of the total reads (roughly 187500) for our analysis.

FIG. 7B shows the distribution of fold enrichment for the 81 different gene sets encoding a virtual peptide sequence in the RRSFV (SEQ ID NO:2) family. Use of the two-fold redundant genetic code makes the hit gene-set enrichments more similar to each other than is the case for individual RRSFV(SEQ ID NO:2)-encoding genes, with the 5%-95% spread in log fold enrichment about three quarters of the value seen in the one codon per amino acid case. The two-fold redundant code also provides excellent detection of hit gene-sets within a ranked list of gene-sets. It is possible to find 58 of the 81 hit gene-sets with a false discovery rate of less than 90%. This is a marked improvement over the non-redundant genetic code, given equal sequencing depth.

A key operational factor in a directed chemical evolution experiment is the sensitivity to detect gene enrichment within a defined chemical space. This depends on the fold enrichment conferred by the selection, the depth of sequencing, and the complexity of the chemical space in question. The three parameters are linked by the fact that a single gene must appear at least twice in the sequencing data to be distinguished from noise. For example, our library was comprised of 20 billion genes and we collected three million reads per sequencing run. Thus, single genes had to be enriched 15000-fold on average to generate an interpretable enrichment signal. This required 3-4 generations.

The relationship between gene enrichment and the true selection fitness of an encoded small molecule is key factor. Accurate knowledge of selection fitness is important, because synthesizing and testing putative hits one at a time is a resource-limiting step. A poor correlation between enrichment and fitness leads to wasted effort on sub-optimal compounds, and could mean that the very best molecules in a library would be missed. The examples provided here illustrate several ways to improve enrichment-fitness correlation: increasing sequencing depth, averaging over synonymous gene sequences via a redundant genetic code, and breeding over more generations.

The data indicate that directed chemical evolution, when coupled with next generation DNA sequencing, can provide quantitative structure-activity relationships for an entire library. The microplate format of our approach is compatible with hundreds-to-thousands of chemical building blocks. Evolution of large-alphabet libraries will empower scientists to explore uncharted areas of chemical space and generalize the notion of natural products. Applications include new functional materials, biological effectors and probes, and small-molecule tools for industrial processes. In short, directed chemical evolution promises to deliver molecules that solve a number of expensive and currently intractable problems.

It will also be recognized by those skilled in the art that, while the invention has been described above in terms of preferred embodiments, it is not limited thereto. Various features and aspects of the above described invention may be used individually or jointly. Further, although the invention has been described in the context of its implementation in a particular environment, and for particular applications those skilled in the art will recognize that its usefulness is not limited thereto and that the present invention can be beneficially utilized in any number of environments and implementations. Accordingly, the claims set forth below should be construed in view of the full breadth and spirit of the invention as disclosed herein.

What is claimed is:

1. A method of screening, comprising:
a) combining a nucleic acid-programmed library with:
(i) an enzyme; and
(ii) a substrate for the enzyme;
wherein each library member comprises a test agent that is linked to an nucleic acid tag that encodes the test agent and where the enzyme covalently transfers a chemoselective functional group from said substrate to one or more library members;
b) isolating the library members onto which said chemoselective functional group has been covalently transferred; and
c) amplifying the nucleic acid tags of the library members isolated in step b) to produce an amplification product,
wherein the method optionally comprises:
d) optionally sequencing members of c); and
e) optionally iterating steps a-d.

2. The method of claim 1, wherein the nucleic acid tags of the library members isolated in step b) are optionally recombined with one another to produce new nucleic acid tags.

3. The method of claim 1, wherein said method further comprises:
d) making a second nucleic acid-programmed small molecule library using the amplification product of c) or diversified progeny of product c) generated by mutation of members of c) and/or by recombination between members of c);
e) combining the second nucleic acid-programmed small molecule library with:
(i) said enzyme, and
(ii) a substrate for the enzyme;
wherein where the enzyme covalently transfers a chemoselective functional group from said substrate to one or more library members;
f) isolating the library members onto which said chemoselective functional group has been covalently transferred in step e); and
g) amplifying the nucleic acid tags of the library members isolated in step f).

4. The method of claim 3, comprising successively repeating steps d) to g) more than one time.

5. The method of claim 3, comprising sequencing the nucleic acid tags of the library members, thereby identifying test agents with covalently attached chemoselective functional groups.

6. The method of claim 1, wherein the enzyme covalently transfers a thiol group from said substrate to one or more library members.

7. The method of claim 6, wherein said substrate is gamma-thio-ATP.

8. The method of claim 1, wherein the enzyme covalently transfers a dipolarophile or a dipolar moiety group from said substrate to one or more library members;

9. The method of claim 1, wherein the method further comprises reacting said chemoselective functional group with a capture molecule, and isolating the library members with covalently attached chemoselective functional groups using a solid support that binds to a capture moiety of said capture molecule.

10. The method of claim 1, wherein the enzyme is a kinase.

11. The method of claim 1, wherein nucleic acid-programmed small molecule library has a complexity of at least 10³.

12. The method of claim 1, wherein the test agents in the library are at least 4 residues in length.

13. A method comprising:
a) making a nucleic acid-programmed small molecule library that comprises a first set of members and a second set of members, wherein the first set of members and the second set of members are essentially identical except for a tag that allows said first and second sets of members to be separated by hybridization; and
b) separating the first and second sets by hybridization.

14. The method of claim 13, further comprising:
c) screening said first set of library members under a first set of conditions to obtain first results;
d) screening said second set of library members under a second set of conditions to obtain second results; and
e) comparing the results obtained from steps c) and d), and
f) optionally selecting library small molecules for further work based on the comparison.

15. A composition comprising:
a) a first set of members of a nucleic acid-programmed small molecule library; and
b) a second set of members of a nucleic acid-programmed small molecule library,
wherein the first and second sets of members of the library are essentially identical except for a tag that allows said first and second sets of members to be separated from one another by hybridization.

16. A compound selected from the group consisting of: RRSFL (SEQ ID NO:1), RRSFV (SEQ ID NO:2), RRASL (SEQ ID NO:3), RRFSV (SEQ ID NO:4), RRMSV (SEQ ID NO:5), RRMTV (SEQ ID NO:6), RMSF (SEQ ID NO:7), RRSF (SEQ ID NO:8) and RRMS (SEQ ID NO:9).

17. A pharmaceutical composition comprising the compound of claim 16 and a pharmaceutically acceptable excipient.
